(19) United States
(12) Patent Application Publication
(10) Pub. No.: US 2009/0156687 A1
(43) Pub. Date: Jun. 18, 2009

(54) Fab I and inhibition of apicomplexan parasites

(76) Inventors: Rima McLeod, Chicago, IL (US); Michael Kirisits, Ho Chi Minh City (VN); Dennis Kyle, Lithia, FL (US); Douglas G. Mack, Centinnel, CO (US); Wilbur Milhous, Tampa, FL (US); Stephen Muench, Leeos (GB); Ernest J. Mui, Chicago, IL (US); Sean Prigge, Severna Park, MD (US); John Rafferty, Sheffield (GB); David Rice, Sheffield (GB); Craig W. Roberts, Glasgow (GB); Benjamin Samuel, Chicago, IL (US)

Correspondence Address: MS. ELIZABETH ARWINE, ESQ.;USMRMC-OSJA, 504 SCOTT STREET, FREDERICK, MD, 21702, US

(21) Appl. No.: 11/998,988

(22) Filed: Nov. 5, 2007

Related U.S. Application Data
(63) Continuation of application No. 10/465,527, filed on Jun. 18, 2003, now abandoned.

(51) Int. Cl. A61K 31/09; C12N 9/02; C07K 14/00; C12Q 1/26; C12Q 1/68; A61P 33/02

(52) U.S. Cl. 514/717; 435/189; 530/350; 435/25; 435/6

(57) ABSTRACT

Discovery and characterization of an apicomplexan Fab I gene and encoded enzyme and discovery of the triclosan as a lead compound, provide means to rationally design novel inhibitory compositions useful for prevention and treatment of apicomplexan related diseases.

This application is a continuation application of, and claims priority to, previously filed U.S. Ser. No. 10/465,527, filed Jun. 13, 2003, entitled “Fab I and Inhibition of Apicomplexan Parasites,” the contents of which are expressly incorporated by reference into the present application.

### BACKGROUND

Discovery and characterization of an apicomplexan Fab I gene and encoded enzyme and the discovery of triclosan as a lead compound, provide means to rationally design novel inhibitory compositions useful for prevention and treatment of apicomplexan and microbial related diseases.

Fab I, enoyl acyl carrier protein reductase (ENR), is an enzyme used in fatty acid synthesis. It is a single chain polypeptide in plants, bacteria, and mycobacteria, but is part of a complex polypeptide in animals and fungi. Certain other enzymes in fatty acid synthesis in apicomplexan parasites appear to have multiple forms, homologous to either a plastid sequence, a plant-like single chain enzyme, or more like the animal complex polypeptide.

Apicomplexan infections are among the most common and devastating infectious diseases. Malaria (Plasmodium) kills one child every eleven seconds and three million people every year. It is a cause of substantial morbidity in pregnant women and young children. Toxoplasmosis gondii results in a chronic central nervous system infection in more than a third of the world population, as well as acute life threatening disease in immunocomprised individuals. New medicines are greatly needed for the treatment of these diseases.

Recently a number of plant-like biochemical pathways associated with the vestigial plastid organelle of T. gondii and Plasmodium species have been suggested as new targets for such medicines (Roberts et al, 1998; Waller et al., 1998; Zuther et al., 1999). A particularly attractive target in this respect is the fatty acid biosynthesis pathway because there are major differences between the structure of the plastid-associated enzymes found in plants and the cytosolic enzymes found in mammals (Roberts et al., 1998; Jomaa et al., 1999; Zuther et al., 1999; Waller et al., 2000). Importantly, enzymes of mammalian lipid synthesis form domains on a multi-functional protein, whereas those enzymes in plants and certain bacteria are found on discrete mono-functional polypeptides. These differences have already been exploited by a number of compounds which selectively inhibit bacterial or plant enzymes, but do not inhibit mammalian enzymes (Roberts et al., 1998; Waller et al., 1998; Zuther et al., 1999; Payne et al., 2000). Notably, both T. gondii and P. falciparum have been shown to possess mono-functional, plant- or bacterial-like fatty acid biosynthesis enzymes which are targeted to the plastid organelle via a bipartite, N-terminal transit sequence (Waller et al., 1998; Zuther et al., 1999; Roos et al., 1999; DeRocher et al., 2000). Compounds such as aryloxyphenoxypropionates (Zuther et al., 1999), cyclohexanedione (Zuther et al., 1999) herbicides and thiolactomycin (Waller et al., 1998) which inhibit acetyl-CoA carbozylase (ACC) and (3-ketoacyl-ACPsynthase (Fab H) respectively, have been demonstrated to restrict the growth of T. gondii in vitro.

Enoyl acyl carrier protein reductase catalyses the NAD (P)-dependent reduction of a trans-2,3 enoyl moiety into a saturated acyl chain, the second reductive step in the fatty acid biosynthesis pathway. Recent studies on the inhibition of ENR by compounds such as the diazaborines (Turnowsky et al., 1989; Baldock et al., 1996) and triclosan (McMurray et al., 1998; Heath et al., 1998; Levy et al., 1999; Payne et al., 2000; and Jones et al., 2000) have validated this enzyme as a target for the development of new antibacterial agents. In particular, triclosan, which is found in many house-hold formulations including soaps, deodorants, hand lotion, toothpaste and impregnated into plastics as an antibacterial agent is an extremely potent ENR inhibitor (Ward et al., 1999). A question is whether Fab I is in apicomplexan parasites and, if so, whether inhibition of Fab I can inhibit parasite growth and/or survival.

### SUMMARY OF THE INVENTION

The present invention relates the first report of apicomplexan Fab I (enoyl acyl carrier protein reductase, ENR) and discloses the effects of triclosan, a potent and specific inhibitor of this enzyme, on the in vitro growth of T. gondii and P. falciparum chain. A plant-like Fab I in P. falciparum was identified by the inventors and the structure was modeled on the Brassia napus and Escherichia coli structures, alone and complexed to triclosan (5-chloro-2-[2,4 dichloropheoxyl] phenol), which confirmed all the requisite features of an enoyl acyl carrier protein reductase (ENR) and its interactions with triclosan. Like the remarkable effect of triclosan on a wide variety of bacteria, this compound markedly inhibits growth and survival of the apicomplexan parasites P. falciparum and Toxoplasma gondii at low concentrations (i.e., IC50=150-2000 and 62 nanogram/ml respectively).

Initially, a sequence for a putative Plasmodium falciparum Fab I was located on the aggregate P. falciparum chromosomes referred to as “blob” (GenBank Accession Number AF338731). The deduced amino acid sequence and a multisequence alignment with representative enoyl acyl carrier protein reductases are shown in FIG. 1 (GenBank Accession Number AF33781). GenBank web site is www.ncbi.nlm.nih.gov. The gene sequence of Plasmodium ENR was obtained with a BLAST search using the sequences from both the B. napus and E. coli enzymes within the P. falciparum database “PlasmoDB” (found at www.PlasmoDB.org). (See Materials and Methods). This sequence was then converted to an amino acid sequence at www.expasy.ch/tools/dna.html. The sequence was aligned using the “Multiple Sequence Alignment at http://searchlauncher.bcm.tmc.edu.

Subsequently, the molecules were prepared and tested in a laboratory setting (see Example 1). Errors in the published sequence for the Plasmodium genome were found. FIG. 1 shows the correct amino acid sequence for Plasmodium.

Analysis of the pattern of sequence conservation confirmed that this protein has all the residues that have been identified as essential for enzyme activity. Interestingly, there is much greater sequence similarity with the plant enzyme than with the ENRs of bacterial origin. The P. falciparum enoyl acyl carrier protein reductase appears to have a plastid targeting sequence (Waller et al., 2000) and has a number of internal insertions. In addition, the P. falciparum protein has an extremely polar additional internal insertion for which no counterpart exists in any of the previously described enoyl acyl carrier protein reductases. This is important to target sequences among that are unique species of Fab I targets that can be attacked with antisense.

Because Fab I was located, the effects of triclosan on Plasmodium falciparum in vitro were investigated. For P. falciparum, the in vitro assays (Milhous et al., 1985; Oduola et al., 1988) were conducted using a modification of the semiautomated microdilution technique for assessing anti-folate antagonists. Instead of dialysed human plasma, 10% Albumax I (Gibco BRL), a serum-free substitute, was used to supplement the RPMI 1640 medium. All test compounds were dissolved in DMSO and diluted 400-fold into complete medium before serial dilution over 11 concentrations. Incubation was at 37° C. in 5% O2, 5% CO2 and 90% N2 for 48 h. [3H]-Hypoxanthine incorporation was measured as described previously (Milhous et al, 1985; Oduola et al, 1988). P. falciparum strain W2 is susceptible to mefloquine, but resistant to pyrimethamine, sulphadoxine and quinine and less susceptible to chloroquine than P. falciparum strain D6. Strain D6 is susceptible to pyrimethamine and sulphadoxine, but similar to P. falciparum strains TM90C2A and TM90C2B, and strain TM91C235 is less susceptible to mefloquine.

The effect of triclosan on P. falciparum in vitro was studied with pyrimethamine sensitive and resistant organisms, and those with varying sensitivity to chloroquine and mefloquine, simultaneously with studies of effect of chloroquin or mefloquine on these parasites (Table 1). Triclosan was effective against pyrimethamine resistant P. falciparum (W2) at low concentrations (IC50s of 150 nanograms/ml [triclosan] and 160 ngm/ml [Chloroquin], respectively) (Table 1). Interestingly, the pattern of relative susceptibility of triclosan and mefloquine were identical. This similarity suggests that triclosan and mefloquine may share a common mechanism of influx or efflux, because such differences in transporters are believed to be the basis of the differences in susceptibility of malaria parasites to mefloquin although other mechanisms are possible.

For T. gondii, growth inhibition was assessed over a 4-day period as described previously (Mack et al., 1984; Roberts et al., 1998; Zuther et al., 1999) using human foreskin fibroblasts (HFF) infected with 105 tachyzoites of the RH strain of T. gondii. The assays are based on microscopic visual inspection of infected and inhibitor treated cultures, and on quantitation of nucleic acid synthesis of the parasite by measuring uptake of 3H uracil into the parasite's nucleic acid. Uracil is not utilized by mammalian cells. Parasites present as tachyzoites (RH, Ptg., a clone derived from the Me49 strain), bradyzoites (Me49), and R5 mutants (mixed tachyzoites/bradyzoites of the Me49 strain that can be stage switched by culture conditions) (Bohne et al., 1993; Soete et al., 1994; Tomovo and Boothroyd, 1995; Weiss et al., 1992) are suitable for assay systems used to study effectsof inhibitors. Only the RH strain tachyzoites, cultured for up to 72 hours, had been used in previously reported assays. The use of Me49, Ptg, and R5 mutants are unique aspects of the methods used in these assays in this invention.

Results using the assay systems are shown in FIGS. 6-8. In these assays toxicity of a candidate inhibitor was assessed by its ability to prevent growth of human foreskin fibroblasts (HFF) after 4 days and after 8 days as measured by tritiated thymidine uptake and microscopic evaluation. Confluent monolayers of HFF were infected with tachyzoites and bradyzoites. Inhibitor was added one hour later. Non-toxic doses were used in parasite growth inhibition assays. Parasite growth was measured by ability to incorporate tritiated uracil during the last 48 hours of culture.

Triclosan also was effective against T. gondii, in nanomolar amounts (FIG. 2). IC50 was 62 nanograms/ml. There was no toxicity to host cells at these concentrations.

Analysis of the binding site for triclosan in B. napus and E. coli ENR shows that 11 residues have contacts less than 4 A with one of more atoms of the triclosan (FIG. 3). Inspection of the sequence for P. falciparum ENR reveals that it shares sequence identity at each of these positions with either the sequence of the B. napus or E. coli enzymes providing a clear explanation for the inhibitory properties of this agent against P. falciparum.

The discovery and characterization of an apicom-plexan Fab I and discovery of triclosan as a lead compound provide means to rationally design novel inhibitory compounds with considerable promise. The invention provides novel ways to counteract the increasing resistance of Plasmodium to the current armoury of antimalarial agents and provides a new approach to the great need for additional, less toxic antimicrobial agents effective against T. gondii. The inventors (Zuther et al., 1999) and others (Waller et al., 1998) have also identified other novel inhibitors of sequential enzymatic steps in the apicomplexan lipid synthesis pathway, that are predicted to be synergistic with triclosan and other inhibitors of Fab I (Baldock, et al., 1996). This also raises the exciting possibility of a rational basis for discovery of synergistic inhibitors of this pathway effective against multiple different microorganisms (Payne et al., 2000).

### BRIEF DESCRIPTION OF THE DRAWINGS

FIG. 1 shows a multiple structure-based sequence alignment of the enoyl reductases from E. coli, H. pylori, B. subtilis, S. aureus, P. falciparum and B. napus. The secondary structures and sequence numbers of the E. coli and B. napus enzymes are shown above and below the alignment, respectively. The residues which are completely conserved are all black with white faced type and those involved in triclosan binding are indicated with a black, filled circle above. The N terminal sequence in the P. falciparum Fab I with no corresponding sequence in E. coli is a plastid target sequence which is a suitable separate target from the entire enzyme for inhibition.

FIG. 2 demonstrates inhibition of T. gondii by triclosan. (a) no inhibitory effect of triclosan on the host cells uptake of thymidine; appearance of the monolayer also was unchanged, (b) effect of triclosan on T. gondii uracil uptake; triclosan reduces uracil uptake by intracellular T. gondii 4 days after infection; IC50 was =62 nanograms per ml; effect increased between days 1 to 4, for example, in a separate experiment, for 125 nanograms per ml of triclosan on day 1, percentage inhibition was 20% and on day 4 was 72% and for pyrimethamine/sulfadiazine percentages of inhibition at these times were 63% and 100% respectively. Abbreviations: RH=RH strain of T. gondii within fibroblasts; No RH=control with fibroblasts alone; DMSO=fibroblasts with highest concentration of DMSO; P/S=fibroblasts, T. gondii, pyrimethamine and sulfadiazine used as a positive control for the assay; CPM=counts per minute.

FIG. 3 is a stereo view of the three dimensional arrangement of the atoms that form the binding pocket for triclosan, in E. coli enoyl reductase, with the 11 residues that have any atom within 4 A of the inhibitor, labeled. This is important in assigning the relative contributions made to the interaction with triclosan by the critical amino acids that are also present in the P. falciparum enzyme.

FIG. 4 shows FabIt fusion protein cut with Factor Xa protease (lane 2) was applied to a cation exchange column (SP Sepharose) separating the fusion protein (lane 3) from Fab It (lanes 4-10). Molecular weight markers (Sigma Wide) are shown in lane 1.

FIG. 5 shows the expression and purification of recombinant Fab I. Fabl-MBP fusion protein cut with Factor Xa protease (lane 2) was applied to a cation exchange column (SP Sepharose) separating MBP (lane 3) from Fab I (lanes 4-10). A small amount of uncut fusion protein (Fabl-MBP) can be seen in the elution fractions as well as some lower molecular weight fragments resulting from overdigestion of Fab I.

FIG. 6(A) is a schematic representation of the pathway for conversion of shikimate to chorismate in T. gondii. The inhibitor of EPSP synthase is NMPG; (B) shows uptake of tritiated uracil by tachyzoites (RH strain) is inhibited by NPMG. Toxicity of NPMG was assessed by its ability to prevent growth of human foreskin fibroblasts (HFF) after 4 days, as measured by tritiated thymidine uptake and microscopic evaluation; (C) shows product rescue of NPMG's inhibitory effect of EPSP synthase on PABA. The effect of PABAon sulfadiazine is similar, but the effect of pyrimethamine, as predicted reduces the enzyme to the levels that were present when media alone was utilized, as measured by the uracil uptake. S=sulfdiazine; PYR=pyrimethamine; and PABA=para amino benzoic acid; (D) shows functional and enzymatic evidence for the shikimate pathway in T. gondii with inhibition of EPSP synthase enzyme activity by 1 mM glyosate. Squares, without glyphosate. Circles, with glyphosate; (E) shows evidence for the shikimate pathway in P. falciparum with functional evidence for the shikimate pathway in P. falciparum. Glyphosate inhibition of in vitro growth of asexual erythrocytic forms and PABA and folate antagonism of growth inhibition. Effect of NPMG on C. parvum was not abrogated by PABA. This suggests that either uptake of PABA by C. parvum differs or effect of NPMG is on a different branch from the shikimate pathway in C. parvum.

FIG. 7 shows inhibitory effects of NPMG, gabaculine, SHAM 8-OH-quinoline and on Cryptosporidia. 3NPA also inhibited Cryptosporidia.

FIG. 8 shows the effect of NPMG, pyrimethamine, and pyrimethamine plus NPMG on survival of mice following intraperitoneal infection with 500 tachyzoites of the RH strain of T. gondii. Dosage of NPMG was 200 mg/kg/day and pyrimethamine was 12.5 mg/kg/day.

FIG. 12 shows acetyl Co-A carboxylases of apicomplexan were identified. T. gondii was ionhibited by the herbicide, clodinafop, 1 micromolar. A and C control; B and D with clodinafop.

FIG. 13 shows a model of triclosan binding to its target enzyme, ENR.

FIG. 14 shows the fatty acid synthesis pathway.

FIG. 15 is the molecular formula and model for triclosan.

### DETAILED DESCRIPTION OF THE INVENTION

A plant-like FAB I was identified in Plasmodium falciparum. The nucleotide sequence and deduced amino acid sequence was prepared and correct sequences were confirmed. FAB I is a single chain, discrete enzyme. All requisite residues for FAB I enzyme activity were confirmed. The P. falciparum enayl acyl carrier protein reductase has a putative plastid targeting sequence and unique polar insertions. The FAB I structure is modeled on E. coli and B. napus FAB I structure alone and complexed to triclosan. Key amino acids were identified for 2° structure. Residues for binding triclosan were conserved providing explanation for inhibition by triclosan. Triclosan inhibits P. falciparum, T. gondii (nm) in a pattern similar to the action of mefloquine. Soluble protein can be overexpressed.

Information obtained from P. falciparum because FAB I was purified include that the N terminal sequence is the same as B. napus FAB I, enzyme activity is NADH dependent and inhibited by triclosan. FAB I is involved in synthesis of 10, 12 C fatty acids. In a P. berghei murine model, Triclosan administered subcutaneously (3 or 38 mg/kg) was nontoxic, cleared parasitemia and prevented death. Synergy was demonstrated in vitro with cerulein, an inhibitor of Fab F, B, H.

### Materials and Methods

T. gondii in vitro. Growth inhibition was assessed over a 4-day period as described previously by Roberts et al., 1998; Zuther et al., 1999; and Mack et al., 1984, all incorporated by reference, using human foreskin fibroblasts (HFF) infected with 105 tachyzoites of the RH strain of T. gondii. Uptake of 3H uracil was determined. Evaluation of slides of preparations containing HFF, Toxoplasma and inhibitors were made.

P. falciparum. The in vitro assays (Oduala et al., 1988; Milhous et al., 1985) were conducted using a modification of the semiautomated microdilution technique for assessing antifolate antagonists. Instead of dialysed human plasma, 10% Albumaz I (Gibco BRL), a serum-free substitute, was used to supplement the RPMI 16-40 media. All test compounds were dissolved in DMSO and diluted 400-fold into complete media before serial dilution over 11 concentrations. Incubation was at 37° C. in 5% O2, 5% CO2 and 90% N2 for 48h, [3H]-Hypoxanthine incorporation was measured as described previously (13, 14). W2 is susceptible to mefloquine, but resistant to pyrimethamine, sulpha- doxine, but similar to TM90C2A and TM90C2B, and TM91C235 is less susceptible to mefloquin.

TABLE 1
IC 50¹ OF Triclosan, Chloroquine, AND Mefloquine When
Cultured with P Falciparum (Nanograms/Ml)

| Antimicrobial | Parasite Strain | | | | |
|---|---|---|---|---|---|
| Agents | D6 | TM90C2A | W2 | TM90C2B | TM91C234 |
| Triclosan | 387.1 | 1891.4 | 154.4 | 1330.4 | 1800.5 |
| Mefloquine | 5.3 | 24.5 | 2.0 | 19.3 | 19.6 |
| Chloroquine | 3.8 | 57.3 | 162.4 | 82.7 | 46.1 |

¹The activity of triclosan, mefloquine, and chloro-quine were tested against a series of P. falciparum isolates and clones with differing susceptibilities to antimalarial drugs. D6, a clone from the African Sierra I/UNC isolate, is chloroquine and pyrimethamine susceptible; W2 is a clone of the Indochina I isolate and is chloroquine and pyrimethamine resistant. TM90C2A, TM90C2B, and TM91C235 are isolates from Thailand and all are chloroquine and mefloquine resistant. TM91C235 was isolated from a patient that failed mefloquine twice, whereas TM90-C2a and TM90-Cb are admission and recrudescent isolates, respectively, of the first patient who failed treatment with atovaquone (alone) in Thailand. Subsequent susceptibility testing demonstrated that the recrudescent isolate (2B) was approximately 2000 fold resistant to atovaquone, when compared with the admission isolate and other atovaquone - susceptible isolates from Thailand.

Cloning of the FabI gene. The FabI gene from Plasmodium falciparum is located on chromosome 4 and codes for a 432 amino acid protein. The FabI gene from gDNA of the 3D7 strain of P. falciparum was amplified using Pfu Turbo polymerase (Stratagene) and two primers (5′-GGTGGTGAATTCATGAATAAAATATCACAACGG-3′ and 5′ GGTGGTGTCGACTTATTCATTTTCAT-TGCGATATATATC-3′). The resulting amplicon was digested with EcoRI and Sail endonucleases and gel purified using the QIAquick Gel Extraction Kit from Qiagen. The digested ampicon was ligated with T4 DNA ligase (Boehringer Mannheim) into the pMAL-c2x vector (New England Biolabs) which had previously been digested with the same endonucleases and treated with Shrimp Alkaline Phosphatase (USB). A second construct, lacking FabI residues 1-84, was prepared in the same way using the following two primers: 5′-GGTGGTGAATTCTCAAACATAAA-CAAAATTAAAGAAG-3′ and 5′-GGTGGTGTCGACT-TATTCATTTTCATTGCGATATATATC-3′. This truncated construct was called FabIt.

Overexpression of FabIt in bacterial culture. The pMAL-c2x vector containing the FabIt construct was transformed into BL21-CodonPlus(DE3) cells (Stratagene). Bacterial cultures were grown in shaker flasks at 37° to an OD600 of 0.6 and then induced with IPTG (Sigma) to a final concentration of 0.4 mM. Induced cultures were transferred to a 20° shaker and incubated for an additional 12 hours. After this period, the cells were harvested by centrifugation at 5,000×G for 15 minutes and the cell pellet was frozen at −20°.

Purification of Recombinant Fab It fusion protein. Cell lysis buffer (20 mM Na/K phosphate pH 7.5, 1 mg/ml lysozyme (Sigma), 2.5 Dg/ml DNAse I (Sigma), 200 mM NaCl) was added to the frozen cell pellets (20 mL per liter of original culture) and gently vortexed. Resuspended cells were incubated on ice for 10 minutes followed by 30 seconds of sonication. Cell lysate was clarified by centrifugation at 20,000×G for 15 minutes at 4° and applied to a 10 ml amylose column (New England Biolabs) equilibrated in 20 mM Na/K phosphate pH 7.5, 200 mM NaCl. The column was washed with 5 column volumes of 20 mM Na/K phosphate pH 7.5, 500 mM NaCl followed by elution with 20 mM Na/K phosphate pH 7.5, 200 mM NaCl, 100 mM Maltose.

Cleavage of FabIt fusion protein and purification of FabIt. Purified FabIt fusion protein was digested with Factor Xa (New England Biolabs) at ratio of 1 mg Factor Xa per 500 mg of fusion protein. Calcium chloride was added to the reaction mixture at a final concentration of 1 mM and the mixture was incubated at 4° for 24 hours. The reaction mixture was desalted with a HiPrep 26/10 Desalting column (Pharmacia) equilibrated in 20 mM Na/K phosphate pH 8.0, IODM NAD+. Desalted protein was applied to a SP Sepharose cation exchange column (Phaimacia) equilibrated in 20 mM Na/K phosphate pH 8.0, 10DM NAD+ and washed for 10 column volumes with the same buffer. Adsorbed proteins were eluted from the column with a linear gradient to 20 mM Na/K phosphate pH 8.0, 10DM NAD+, 500 mM NaCl in 20 column volumes. Fractions containing pure FabIt protein were pooled for further analysis.

Overexpression of Recombinant Fab I. The FabI gene was amplified from cDNA of the 3D7 strain of P. falciparum and inserted into the pMAL-c2x vector (New England Biolabs) for expression in E. coli. Recombinant FabI fused the Maltose Binding Protein (Fabl-MBP) was purified from clarified cell lysate using a 10 ml amylose column (New England Biolabs). The pure Fabl-MBP fusion protein was cleaved with Factor Xa protease yielding FabI and MBP, which were the separated with a 5 ml SP Sepharose column (Pharmacia).

### DOCUMENTS CITED

Baldock C., Rafferty J. B., Sdelnikova S. E., Baker P. J., Stitje A. R., Slabas A. R., Hawkes T. R., Rice D. W., 1996. A mechanism of drug action revealed by structural studies of enoyl reductase. Science. 274, 2107-2110.

DeRocher A., Hage C. B., Froehlich J. E., Feagin J. E., Parsons M., 2000. Analysis of targeting sequences demonstrates that trafficking to the toxoplasma gondii plas-tid branches off the secretory system. J Cell Sci. 113, (Pt22):3969-77.

Heath R. J., Yu Y. T, Shapiro M. A., Olson E. Rock Colo., 1998. Broad-spectrum antimicrobial biocides target the Fab I component of fatty acid synthesis. J Biol Chem. 273, 30316-30320.

Jomaa H:, Wiesner J., Sanderbrand S., Altincicek B., Weidemeyer C., Hintz M., Turbachova I., Eberl M., Zeidler J., Lichtenhaler H. K., Soldati D., Beck E., 1999. Inhibitors of the nonmevalonate pathway of isoprenoid biosynthesis as antimalarial drugs. Science. 285 (5433):1573-6.

Jones R. D., Janpani H., Newman J. L., Lee A., 2000. Triclosan: A review of effectiveness and safety in healthcare settings. Am J. Inf. Control. 28, 184-196.

Levy, C. W., Roujeinikova A., Sedelnikova S. E., Baker P. J., Stuitje, A. R., Slabas, A. R., Rice, D. W., Rafferty, J. B., 1999. Molecular basis of triclosan activity. Nature. 398, 383-384.

Mack D., McLeod R., 1984. Anew micromethod to study effects of antimicrobial agents on Toxoplasma gondii: Comparison ol sulladoxine and sulladiazine and study of clindamycin, metronidazole, and cyclosporin A. Antimicrob. Agents Chemother. 26, 26-30.

McMurray L. M., Oethinger M., Levy S. B., 1998. Triclosan targets lipid synthesis. Nature. 394, 531-2.

Milhous W. K., Weatherly N. R, Bowdre J. H., Desjardins R. E., 1985. In vitro activities and mechanisms of resistance to anti-folates and anti-malarial drugs. Antimicrob. Agents Chemother. 27, 525-530.

Oduola A. M. J., Weatherly N. J., Bowdre J. H., Desjardins R. E., 1988. Plasmodium falciparum-cloning by single erythrocyte micromanipulation and heterogeneity in vitro. Exp. Parasitol. 66, 86-95.

Payne D. J., Wallis N. G., Gentry D. R., Rosenberg M., 2000. The impact of genomics on novel antibacterial targets. Curr Opin Drug Discovery Devel. 3(2), 177-190.

Plasmodium Genome Database Collaborative, 2001. PlasmoDB: An integrative database of the P. falciparum genome. Tools for accessing and analyzing finished and unfinshed sequence data. Nucl. Acids Res. 29: 66-69.

Roberts R, Roberts C. W., Johnson J., Kyle D. E., Krell T, Coggins J. R., Coombs G. H., Milhous W. K., Tzipori S., Ferguson D. J. P., Chakrabarti D., McLeod R., 1998. Evidence for the shikimate pathway in apicomplexan parasites. Nature. 393, 801-805.

Roos D. S., Crawford M. J., Donald R. G., Kissinger J. C., Klimczak L. J., Striepen B., 1999. origin, targeting, and function of the apicomplexan plastid. Curr Opin Microbiol. 2(4):426-32. Review.

The Plasmodium Genome Database http://Plas-moDB.org (Nucleic Acids Research) 29, In Press 2001.

Tumowsky R, Fuchs K., Jeschek C., Hogenauer G., 1989. env M genes of Salmonella typhimurium and Esc/zen-chia coli. J. Bacteriol. 171, 6555-6565.

Waller R. R, Keeling P. J., Donald R. G., Striepen B., Handman E., Lan-Unnasch N., Cowman A. R, Besra G. S., Roos D. S., McFadden G. I., 1998. Nuclear-encoded proteins target to the plastid in Toxoplasma gondii and Plasmodium falciparum. Proc Natl Acad Sci USA. 95(21), 12352-12357.

Waller R. R, Reed M. D., Cowman A. R, McFadden G. I., 2000. Protein trafficking to the plastid of Plasmodium falciparum is via the secretory pathway. EMBO J.19(8): 1794-802.

Ward W. H. J., Holdgate G. A., Rowesell S., McLean E. G., Pauptit R. A., Clayton E., Nichols W. W, Colls J. G., Minshull C. A., Jude, D. A., Mistry A., Timms D., Gamble R., Hales, N. J., Britton C. J., Taylor, I. W. R, 1999. Kinetic and structural characteristics of the inhibition of enoyl (acyl carrier protein) reductase by triclosan. Biochemistry. 38, 12514-12525.

Zuther E., Johnson J. J., Haselkom R., McLeod R., and Gomicki P., 1999. Growth of Toxoplasma gondi is inhibited by aryloxyphenoxpropionate herbicides targeting acetyl-CoA carboxylase. Proc. Natl. Acad Sci USA. 96, 13387-92.

### SEQUENCE LISTING

Applicants respectfully direct entry of the Sequence Listing entitled “11998988_ST25” into the present application, which is attached in CRF as well as a related printed copy.

We claim:

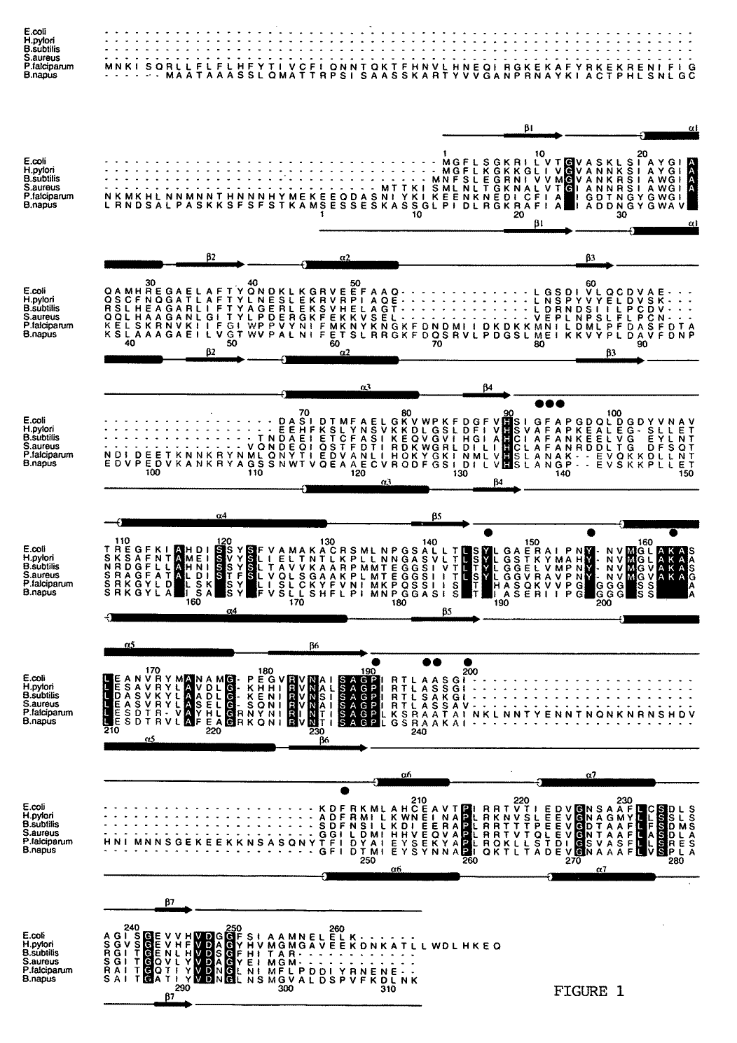

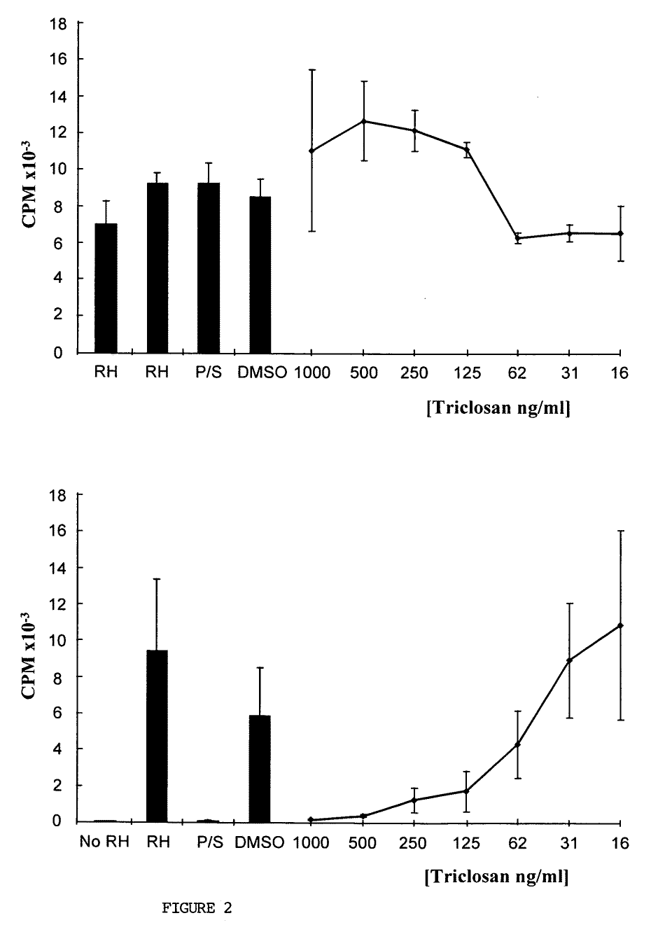

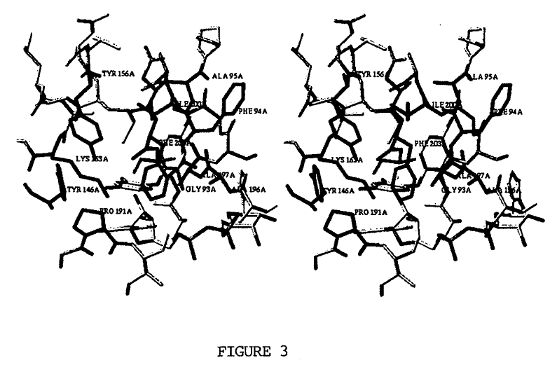

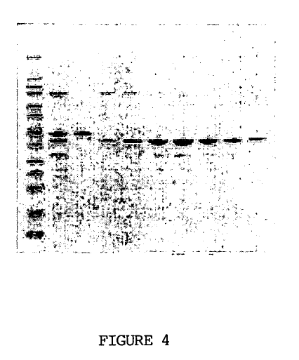

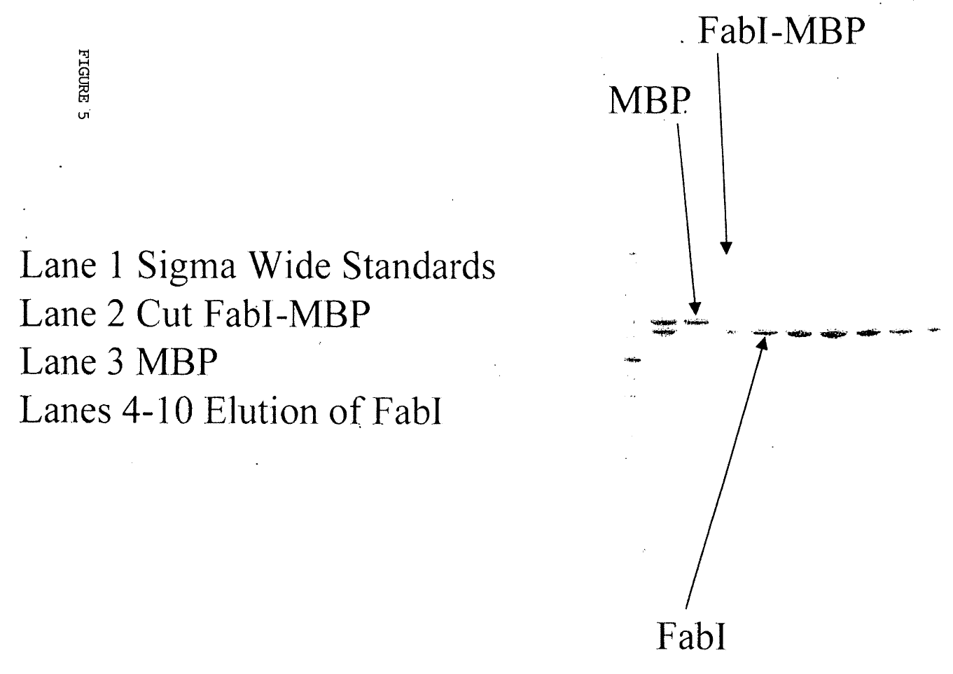

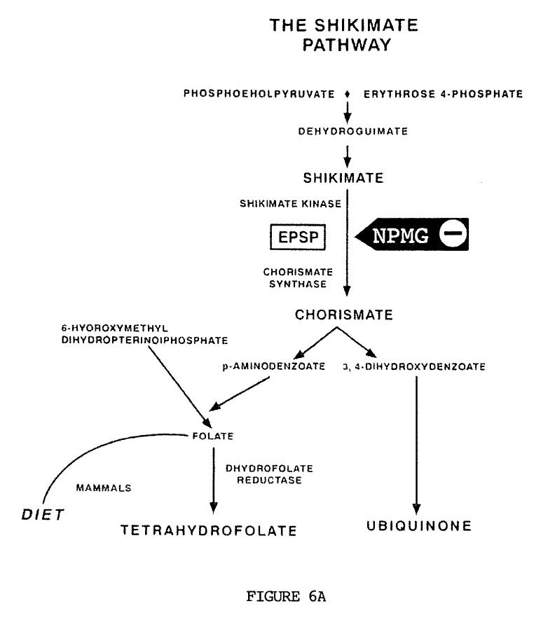

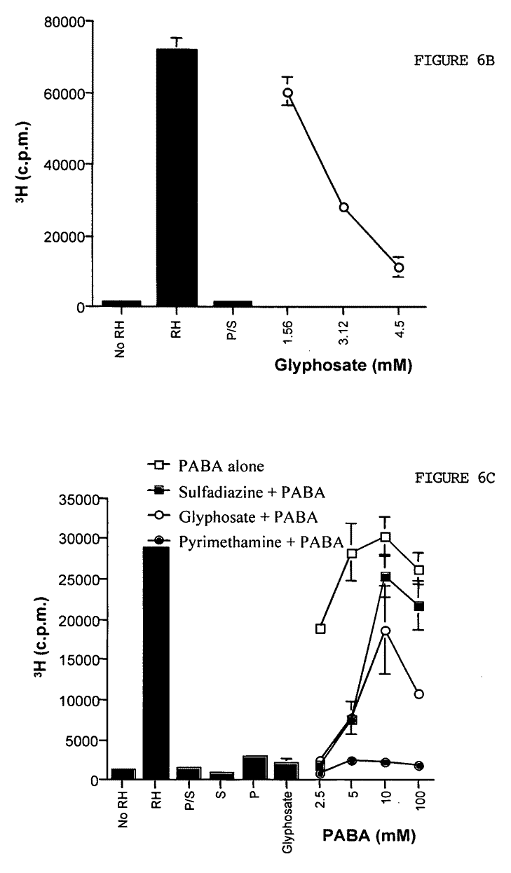

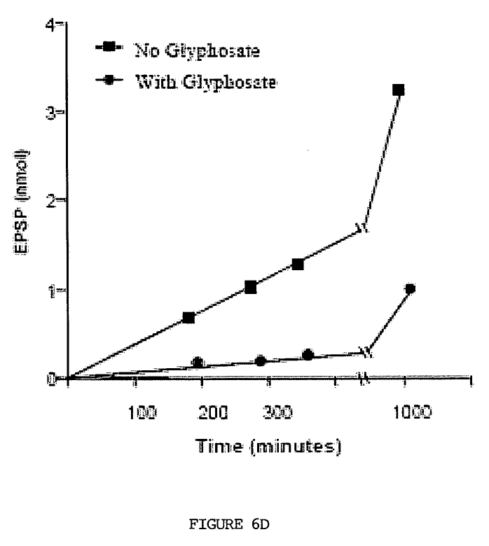

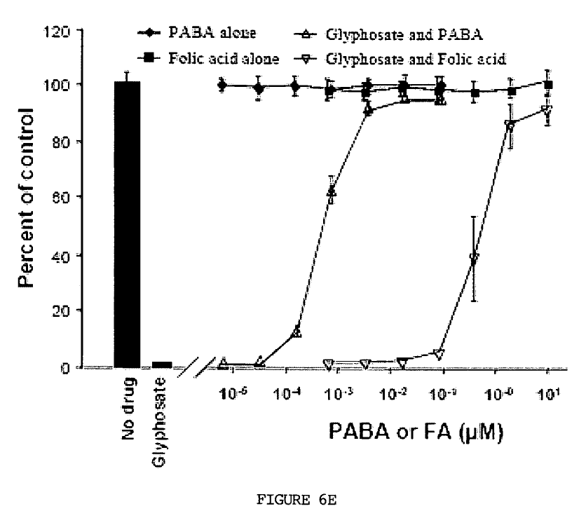

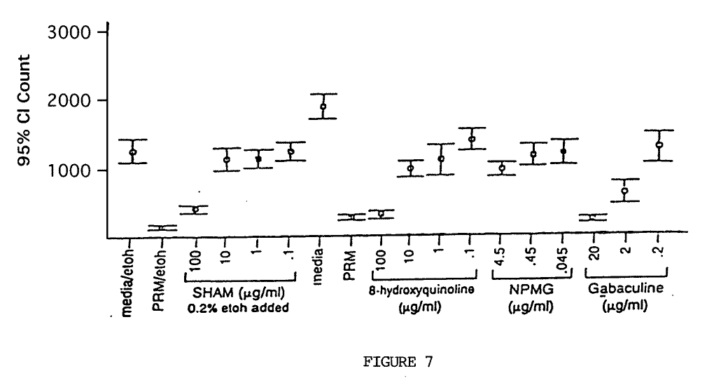

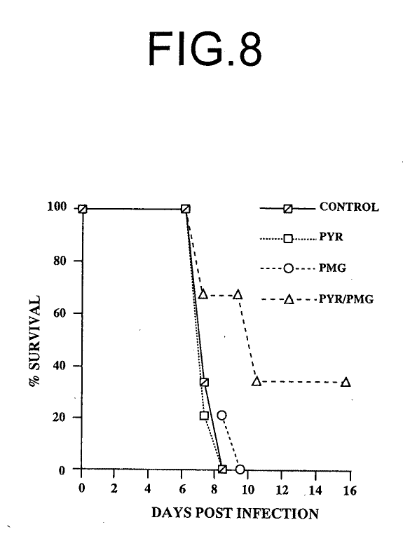

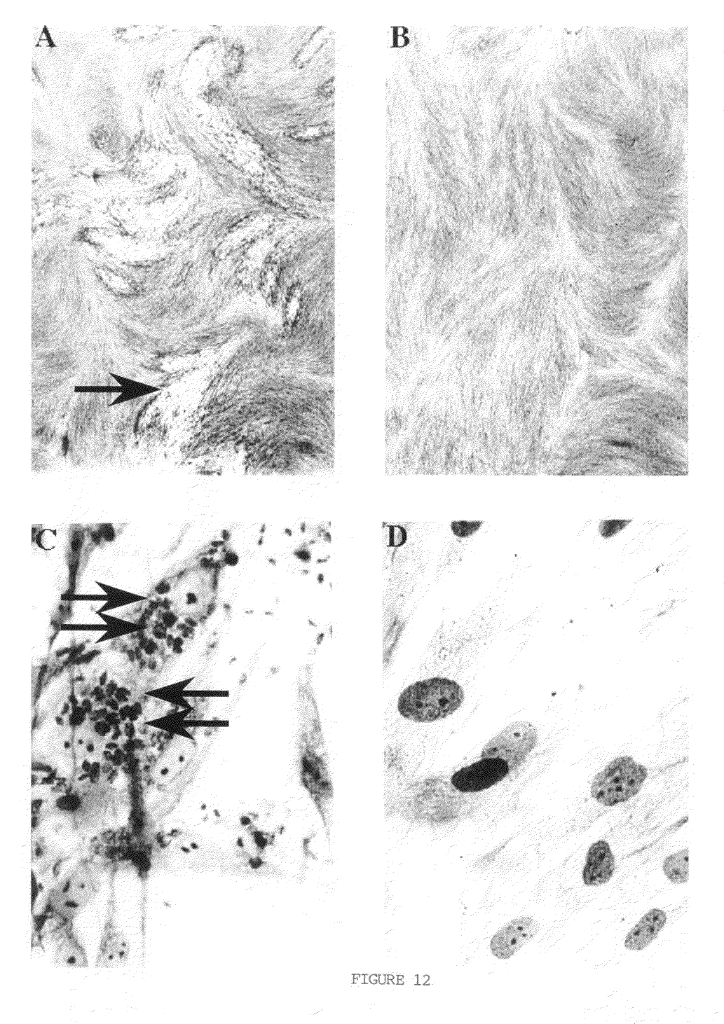

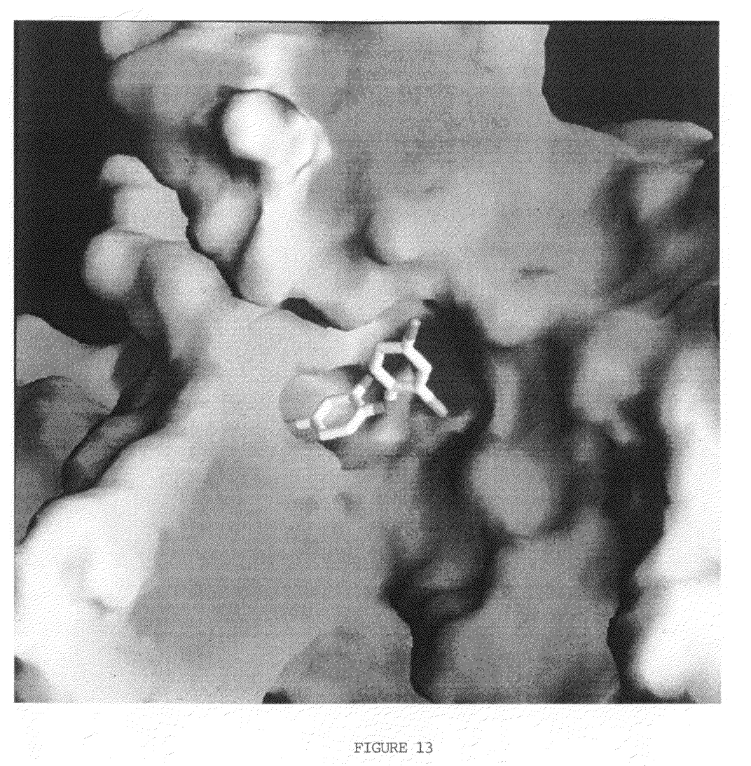

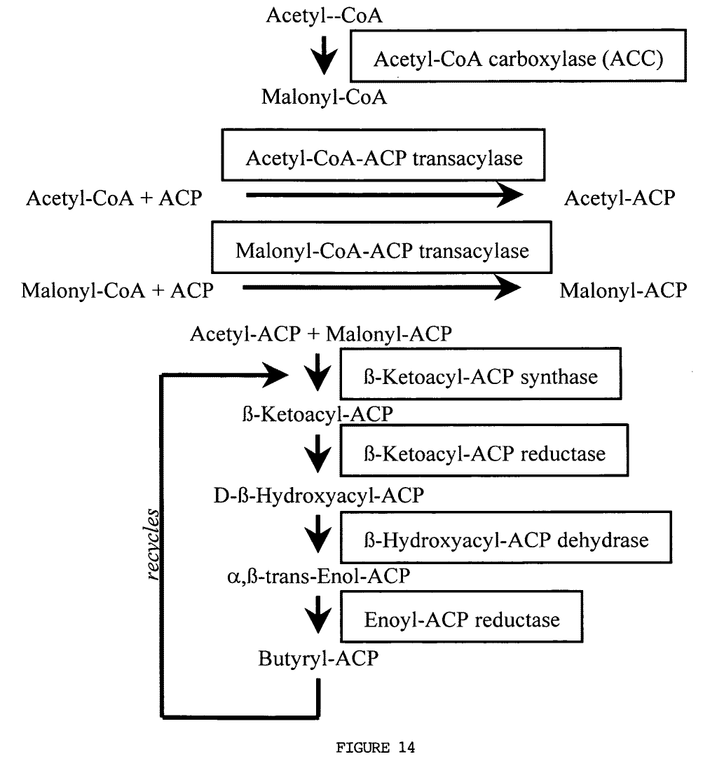

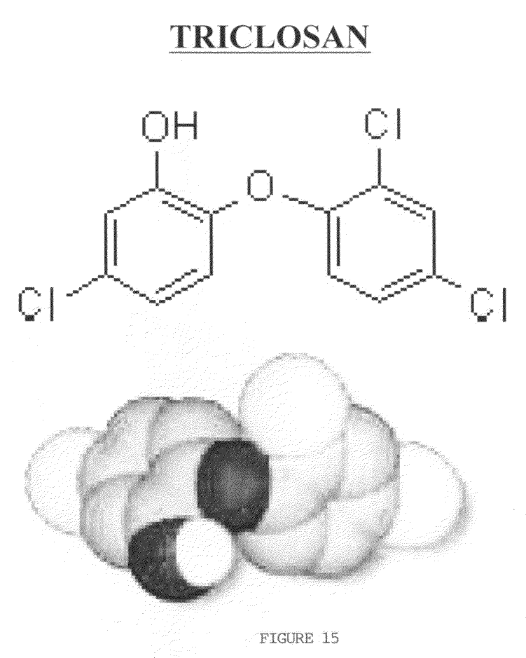

1. A molecule of the fab I enzyme having the amino acid sequence of the Fab I enzyme in Plasmodium falcipamm, as shown in FIG. 1.

2. Use of the amino acid sequence information from apicomplexan Fab I as a target to develop inhibitors and antimicrobal agents disease causing agents.

3. The use of claim 2, wherein the apicomplexan is Plasmodium falcipamm.

4. The use of claim 2 wherein the disease causing agents are bacteria.

5. A novel recombinant protein with an amino acid sequence substantially similar to that of Plasmodium falcipamm shown FIG. 1.

6. Use of the recombinant protein of claim 5 to determine the crystal structure of the enzyme from which novel inhibitors can be designed.

7. Use of the information on the mRNA sequence corresponding to the amino acid sequence of apicomplexan Fab I to develop iRNA which will complete for the FAB I mRNA.

8. Use of the plasmid targeting sequence of the Plasmodium falcipamm Fab I amino acid sequence according to FIG. 1, to design antimicrobial agents and inhibitors of apicomplexan growth and survival.

9. Use of triclosan to inhibit apicomplexan growth and survival.


SEQUENCE LISTING (10 sequences)

SEQ ID NO: 1 - protein, 262 aa, Escherichia coli
MGFLSGKRILVTGVASKLSIAYGIAQAMHREGAELAFTYQNDKLKGRVEEFAAQLGSDIVLQCDVAEDASIDTMFAELGKVWPKFDGFVHSIGFAPGDQLDGDYVNAVTREGFKIAHDISSYSFVAMAKACRSMLNPGSALLTLSYLGAERAIPNYNVMGLAKASLEANVRYMANAMGPEGVRVNAISAGPIRTLAASGIKDFRKMLAHCEAVTPIRRTVTIEDVGNSAAFLCSDLSAGISGEVVHVDGGFSIAAMNELELK

SEQ ID NO: 2 - protein, 275 aa, Helicobacter pylori
MGFLKGKKGLIVGVANNKSIAYGIAQSCFNQGATLAFTYLNESLEKRVRPIAQELNSPYVYELDVSKEEHFKSLYNSVKKDLGSLDFIVHSVAFAPKEALEGSLLETSKSAFNTAMEISVYSLIELTNTLKPLLNNGASVLTLSYLGSTKYMAHYNVMGLAKAALESAVRYLAVDLGKHHIRVNALSAGPIRTLASSGIADFRMILKWNEINAPLRKNVSLEEVGNAGMYLLSSLSSGVSGEVHFVDAGYHVMGMGAVEEKDNKATLLWDLHKEQ

SEQ ID NO: 3 - protein, 258 aa, Bacillus subtilis
MNFSLEGRNIVVMGVANKRSIAWGIARSLHEAGARLIFTYAGERLEKSVHELAGTLDRNDSIILPCDVTNDAEIETCFASIKEQVGVIHGIAHCIAFANKEELVGEYLNTNRDGFLLAHNISSYSLTAVVKAARPMMTEGGSIVTLTYLGGELVMPNYNVMGVAKASLDASVKYLAADLGKENIRVNSISAGPIRTLSAKGISDFNSILKDIEERAPLRRTTTPEEVGDTAAFLFSDMSRGITGENLHVDSGFHITAR

SEQ ID NO: 4 - protein, 264 aa, Staphylococcus aureus
MTTKISMLNLTGKNALVTGIANNRSIAWGIAQQLHAAGANLGITYLPDERGKFEKKVSELVEPLNPSLFLPCNVQNDEQIQSTFDTIRDKWGRLDILIHCLAFANRDDLTGDFSQTSRAGFATALDISTFSLVQLSGAAKPLMTEGGSIITLSYLGGVRAVPNYNVMGVAKAGLEASVRYLASELGSQNIRVNAISAGPIRTLASSAVGGILDMIHHVEQVAPLRRTVTQLEVGNTAAFLASDLASGITGQVLYVDAGYEIMGM

SEQ ID NO: 5 - protein, 420 aa, Plasmodium falciparum
MNKISQRLLFLFLHFYTIVCFIQNNTQKTFHNVLHNEQIRGKEKAFYRKEKRENIFIGNKMKHLNNMNNTHNNNHYMEKEEQDASNIYKIKEENKNEDICFIAIGDTNGYGWGIKELSKRNVKIIFGIWPPVYNIFMKNYKNGKFDNDMIIDKDKKMNILDMLPFDASFDTANDIDEETKNNKRYNMLQNYTIEDVANLIHQKYGKINMLVHSLANAKEVQKKDLLNTSRKGYLDLSKSYLISLCKYFVNIMKPQSSIISTHASQKVVPGGGGSSALESDTRVAYHLGRNYNIRINTISAGPLKSRAATAINKLNNTYENNTNQNKNRNSHDVHNIMNNSGEKEEKKNSASQNYTFIDYAIEYSEKYAPLRQKLLSTDIGSVASFLLSRESRAITGQTIYVDNGLNIMFLPDDIYRNENE

SEQ ID NO: 6 - protein, 374 aa, Brassica napus
MAATAAASSLQMATTRPSISAASSKARTYVVGANPRNAYKIACTPHLSNLGCLRNDSALPASKKSFSFSTKAMSESSESKASSGLPIDLRGKRAFIAIADDNGYGWAVKSLAAAGAEILVGTWVPALNIFETSLRRGKFDQSRVLPDGSLMEIKKVYPLDAVFDNPEDVPEDVKANKRYAGSSNWTVQEAAECVRQDFGSIDILVHSLANGPEVSKKPLLETSRKGYLAISASYFVSLLSHFLPIMNPGGASISTIASERIIPGGGGSSALESDTRVLAFEAGRKQNIRVNTISAGPLGSRAAKAIGFIDTMIEYSYNNAPIQKTLTADEVGNAAAFLVSPLASAITGATIYVDNGLNSMGVALDSPVFKDLNK

SEQ ID NO: 7 - DNA, 33 bp, Artificial Sequence
  Description of Artificial Sequence Synthetic Primer
ggtggtgaattcatgaataaaatatcacaacgg

SEQ ID NO: 8 - DNA, 39 bp, Artificial Sequence
  Description of Artificial Sequence Synthetic Primer
ggtggtgtcgacttattcattttcattgcgatatatatc

SEQ ID NO: 9 - DNA, 37 bp, Artificial Sequence
  Description of Artificial Sequence Synthetic Primer
ggtggtgaattctcaaacataaacaaaattaaagaag

SEQ ID NO: 10 - DNA, 39 bp, Artificial Sequence; identical to SEQ ID NO: 8
  Description of Artificial Sequence Synthetic Primer